(19) United States
(12) Patent Application Publication
(10) Pub. No.: US 2025/0249232 A1
(43) Pub. Date: Aug. 7, 2025

(54) CLINICIAN PROGRAMMER WORKFLOW AUTOMATION FOR IMPLANTABLE MEDICAL DEVICES

(71) Applicant: The Alfred E. Mann Foundation for Scientific Research, Valencia, CA (US)

(72) Inventors: Hitesh Shyamkumar HOTLANI, Santa Clarita, CA (US); Brian Michael SHELTON, Ventura, CA (US)

(21) Appl. No.: 19/046,380

(22) Filed: Feb. 5, 2025

Related U.S. Application Data
(60) Provisional application No. 63/550,544, filed on Feb. 6, 2024.

(51) Int. Cl. A61N 1/02; A61M 31/00; A61N 1/36; A61N 1/372; G16H 10/60; G16H 40/67

(52) U.S. Cl. CPC A61N 1/025; A61M 31/002; A61N 1/36146; A61N 1/37258; A61N 1/37264; G16H 10/60; G16H 40/67; A61M 2205/04; A61M 2205/054; A61M 2205/18; A61M 2205/3523; A61M 2205/52

(57) ABSTRACT

The present disclosure relates to stimulation systems, comprising, e.g., an implantable medical device (IMD), such as an implantable pulse generator configured to deliver electrical stimulation to one or more nerves of a subject, or an implantable pump configured to administer one or more active agents. In some aspects, the IMD is configured to store one or more clinical state parameters (e.g., a clinical workflow stage) and/or to advertise or transmit the stored one or more clinical state parameters to a clinician programmer device. One or more settings of the IMD, and/or parameters of the administration of electrical stimulation or the active agent(s), may be set based on the one or more clinical state parameters (e.g., a clinical workflow stage), e.g., either automatically or based on instructions provided by the clinician programmer device.

### CROSS-REFERENCE TO RELATED APPLICATION

This application claims priority to U.S. Provisional Application No. 63/550,544, filed Feb. 6, 2024, the contents of which is hereby incorporated in its entirety.

### TECHNICAL FIELD

The present disclosure generally relates to systems and methods for managing and automating the provisioning, setup and configuration of implantable medical devices.

### BACKGROUND

Various types of devices have been developed for implantation into the human body to provide health-related therapies and/or monitoring. Examples of such devices, generally known as implantable medical devices (“IMDs”), include cardiac pacemakers, cardioverter/defibrillators, cardiomyostimulators, various physiological stimulators including nerve, muscle, and deep brain stimulators, various types of physiological monitors, and drug delivery systems, just to name a few. For the purposes of this application, particular reference will be made to implantable neurostimulators, such as implantable pulse generators (“IPGs”), it being understood that the principles described herein may be applied to other implantable medical devices as well. IPGs are often used in the context of neuromodulation therapy, and, in general, comprise a hermetically sealed housing containing stimulation circuitry. Electrically conductive feedthrough pins extend from the stimulation circuitry and into a header connector, which is mounted upon the IPG housing, and the header connector defines a receptacle adapted to receive and couple with the proximal end of a stimulation lead.

IMDs are often configured to allow communication with a clinician programming unit (or “clinician programmer device”), a device used by a healthcare professional to program and manage the IMD. Clinician programmer devices may comprise a specialized control unit configured to communicate with and/or configure one or more parameters or settings of a particular type or model of IMD or, in some cases, a general-purpose electronic device (e.g., a computer or server) configured to execute a software application that provides IMD command and/or control functionality. Current clinician programmer devices may allow a healthcare professional to manually connect to an IMD, e.g., via a wireless connection, and to read or modify one or more parameters or settings.

### SUMMARY OF SELECTED ASPECTS

Modern IMDs are complex devices and the initial setup, fitting, and configuration of an IMD is often a time-consuming process for clinicians. Current systems typically require manual setup and configuration and offer little to no options for automation. Manual entry and/or modification of IMD settings is often slow due to the limitations of current interfaces, resulting in an inefficient use of healthcare resources. The novel systems and methods for electronic patient care described herein address this shortcoming, in addition to providing various other benefits as shall be described herein, by providing a convenient workflow for setting up and configuring IMDs. In some aspects, the systems and methods described herein utilize an IMD configured to store one or more clinical parameters (e.g., settings for the IMD and/or parameters for the administration of electrical stimulation or a therapeutic agent), and to transmit or advertise the stored clinical parameters to an associated clinical programmer device. The clinician programmer device may, in turn, set or modify one or more clinical parameters stored in the IMD, or advance a clinical workflow stage, based on user input obtained from a healthcare professional using the clinician programmer device, or automatically (e.g., based on pre-set rules). As used herein, a “clinical workflow stage” refers to an event related to the implantation, testing, fitting, adjustment, repair, or removal of an IMD (e.g., an operation to implant an IMD, an office visit to fit or adjust one or more settings of an IMD, an office visit to test or repair an IMD, an operation to remove an IMD, etc.). Each clinical workflow stage is defined by a set of steps or activities performed to accomplish the stage. For example, a clinical workflow stage for an implantation operation may comprise the activities of: 1) testing the IMD prior to implantation, 2) assigning the IMD to a patient, and then 3) implanting the IMD in the patient. These particular activities represent a non-limiting example; in other aspects, a clinical workflow stage for implantation may comprise alternative or additional activities. It is envisioned that the available clinical workflow stages implemented or recognized by the devices and systems described may be user configurable, and that various options are possible. In some aspects, each clinical workflow stage includes a set of activities or steps associated with or performed during that stage. Aspects of the present embodiments may be implemented throughout the IMD lifecycle, e.g., at the time of implantation, during an initial clinical visit or fitting session, during follow-up clinical visits, and/or for IMD maintenance, promoting increased efficiency and the access to medical care. In some aspects, the clinical workflow stage, and/or the performance of one or more activities thereof, is tracked by a controller of the IMD. This data may be shared, e.g., with a clinician programmer device. For example, a clinician programmer device may be configured to receive data from a controller of an IMD comprising a clinical workflow stage, or one or more clinical state parameters, and to display one or more options to a user via an interface of the clinician programmer device based on the received data (e.g., an option to select or advance a clinical workflow stage on the controller, or to select or modify one or more clinical state parameters stored on the controller). The controller may in turn adjust or set one or more stimulation parameters based on the selected or advanced clinical workflow stage, or the selected/modified clinical state parameters.

In a first general aspect, the disclosure provides a system for electronic patient care, comprising: n implantable medical device (“IMD”) configured to administer (a) electrical stimulation to a nerve of a human subject; and/or (b) an active agent to one or more cells, tissues, or organs of the subject; and a controller, comprising a processor and a memory, and configured to control one or more settings of the implantable stimulator; wherein the controller is configured to store one or more clinical state parameters in the memory, and to allow the one or more clinical state parameters to be read, set, or modified by a clinician programmer device, and to enable, disable, or set, one or more parameters of the administration of the electrical stimulation and/or the active agent, to the subject, based on the stored one or more clinical state parameters.

In some aspects, the one or more clinical state parameters comprise: a) a parameter indicating an IMD battery level or percentage; b) a parameter indicating whether the IMD is currently administering the electrical stimulation to a nerve of a subject; c) a parameter indicating whether the IMD is currently administering the active agent to one or more cells, tissues, or organs of the subject; d) a parameter indicating whether the IMD is implanted in the human subject; e) a parameter indicating a date and/or time of an initial or previous clinical visit by the subject; f) a parameter indicating a date and/or time when the IMD was implanted in the subject; g) a parameter indicating a date and/or time when the subject is due for a subsequent clinical visit; h) a parameter indicating a date and/or time when the subject engaged in a fitting session for the IMD; i) a parameter indicating a date and/or time when the IMD is due for maintenance or replacement; j) one or more parameters indicative of the electrical stimulation currently or previously administered to the subject, by the IMD, wherein the parameters comprise a pulse frequency, width, amplitude, and/or duty cycle, of the electrical stimulation; k) one or more parameters indicative of the active agent currently or previously administered to the subject, by the IMD, wherein the parameters comprise an amount, concentration, dosage schedule or timing, for the administration of the active agent; l) one or more parameters indicative of a location of the IMD; m) one or more parameters indicative of a location, pairing status, availability, or proximity, of a clinician programmer device; and/or n) one or more parameters indicative of a wired or wireless network connection being active or available to the IMD.

In some aspects, the controller is further configured to enable, disable, or set, one or more parameters of the administration of the electrical stimulation and/or the active agent, to the subject, based on the one or more clinical state parameters indicating that a wireless network connection to a clinician programmer device is currently active.

In some aspects, the controller is further configured to enable, disable, or set, one or more parameters of the administration of the electrical stimulation and/or the active agent, to the subject, based on the one or more clinical state parameters indicating that a fitting session for the IMD was previously performed.

In some aspects, the controller is further configured to enable, disable, or set, one or more parameters of the administration of the electrical stimulation and/or the active agent, to the subject, based on the one or more clinical state parameters indicating that the IMD is in proximity to or wirelessly connected to, a clinician programmer device.

In some aspects, the controller is further configured to enable, disable, or set, one or more parameters of the administration of the electrical stimulation and/or the active agent, to the subject, based on the one or more clinical state parameters indicating that the battery level and/or percentage of the IMD is above, below, or within a predetermined range or threshold.

In some aspects, the controller is further configured to generate a text, audio, and/or visual notification that the IMD is in need of maintenance or replacement, based on the one or more clinical state parameters.

In some aspects, the controller is integrated into the IMD, and further configured to transmit data based on or comprising the one or more clinical state parameters, to an external electronic device, and to allow modification of the one or more clinical state parameters, by the external electronic device. In some aspects, the electronic device is a smart phone, a smart watch, a computer, or a portable or stationary external controller.

In a second general aspect, the disclosure provides a method for electronic patient care, comprising: providing an implantable medical device (IMD) configured to administer (a) electrical stimulation to a nerve of a human subject; and/or (b) an active agent to one or more cells, tissues, or organs of the subject, and a controller, comprising a processor and a memory, and configured to control one or more settings of the implantable stimulator; storing, by the controller, one or more clinical state parameters in the memory, and enabling, disabling, or setting, one or more parameters of the administration of the electrical stimulation and/or the active agent, to the subject, based on the stored one or more clinical state parameters.

In some aspects of the methods described herein, the one or more clinical state parameters comprise: a) a parameter indicating an IMD battery level or percentage; b) a parameter indicating whether the IMD is currently administering the electrical stimulation to a nerve of a subject; c) a parameter indicating whether the IMD is currently administering the active agent to one or more cells, tissues, or organs of the subject; d) a parameter indicating whether the IMD is implanted in the human subject; e) a parameter indicating a date and/or time of an initial or previous clinical visit by the subject; f) a parameter indicating a date and/or time when the IMD was implanted in the subject; g) a parameter indicating a date and/or time when the subject is due for a subsequent clinical visit; h) a parameter indicating a date and/or time when the subject engaged in a fitting session for the IMD; i) a parameter indicating a date and/or time when the IMD is due for maintenance or replacement; j) one or more parameters indicative of the electrical stimulation currently or previously administered to the subject, by the IMD, wherein the parameters comprise a pulse frequency, width, amplitude, and/or duty cycle, of the electrical stimulation; k) one or more parameters indicative of the active agent currently or previously administered to the subject, by the IMD, wherein the parameters comprise an amount, concentration, dosage schedule or timing, for the administration of the active agent; l) one or more parameters indicative of a location of the IMD; m) one or more parameters indicative of a location, pairing status, availability, or proximity, of a clinician programmer device; and/or) one or more parameters indicative of a wired or wireless network connection being active or available to the IMD.

In some aspects of the methods described herein, the method further comprises reading, setting or modifying the one or more stored clinical state parameters, by a clinician programmer device.

In some aspects of the methods described herein, such methods further comprise: enabling. disabling, or setting, by the controller, one or more parameters of the administration of the electrical stimulation and/or the active agent, to the subject, based on the stored one or more clinical state parameters indicating that a wireless network connection to a clinician programmer device is currently active.

In some aspects of the methods described herein, such methods further comprise: enabling. disabling. or setting, by the controller, one or more parameters of the administration of the electrical stimulation and/or the active agent, to the subject, based on the stored one or more clinical state parameters indicating that a fitting session for the IMD was previously performed

In some aspects of the methods described herein, such methods further comprise: enabling, disabling, or setting, by the controller, one or more parameters of the administration of the electrical stimulation and/or the active agent, to the subject, based on the stored one or more clinical state parameters indicating that the IMD is in proximity to or wirelessly connected to, a clinician programmer device.

In some aspects of the methods described herein, such methods further comprise: enabling, disabling, or setting, by the controller, one or more parameters of the administration of the electrical stimulation and/or the active agent, to the subject, based on the stored one or more clinical state parameters indicating that the battery level and/or percentage of the IMD is above, below, or within a predetermined range or threshold.

In some aspects of the methods described herein, the controller is integrated into the IMD, and further configured to transmit data based on or comprising the one or more clinical state parameters, to an external electronic device, and to allow modification of the one or more clinical state parameters, by the external electronic device.

In some aspects of the methods described herein, the electronic device is a smart phone, a smart watch, a computer, or a portable or stationary external controller.

In some aspects of the methods described herein, the controller is further configured to wirelessly advertise one or more of the one or more clinical state parameters.

### BRIEF DESCRIPTION OF THE DRAWINGS

FIG. 1 is a block diagram showing an exemplary system according to the disclosure and interactions between the components thereof.

FIG. 2 is a flowchart illustrating an exemplary method according to the disclosure. In this case, the method describes interactions between an IMD (e.g., an implantable electrical stimulation system) and a clinician programmer device.

FIG. 3 is a flowchart illustrating another exemplary method according to the disclosure. In this case, the method illustrates how the present systems may be used to track a clinical workflow stage (or other clinical state parameters), and the modification or advancement of the clinical workflow stages based on instructions received from a clinician programmer device. As noted herein, instructions may be provided as a result of automated (or other) rules and/or user input provided via an interface of a clinician programmer device.

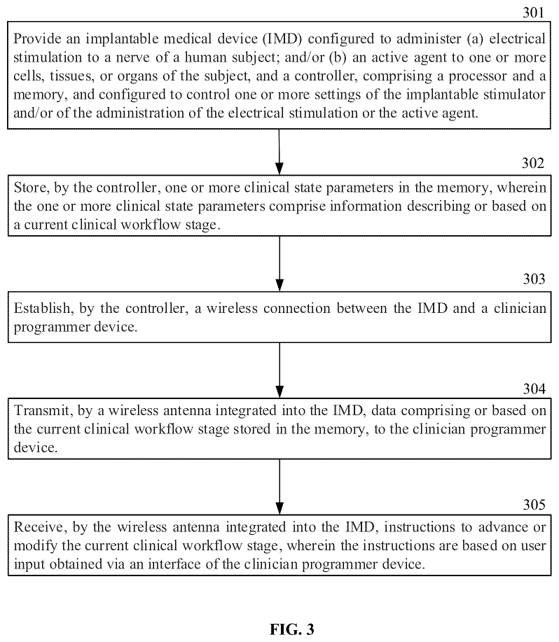

FIG. 4 is a flowchart illustrating another exemplary method according to the disclosure. In this case, the method illustrates the use of automated rules and optional input from a user of a clinician programmer device to advance or modify the current clinical workflow stage stored by an IMD.

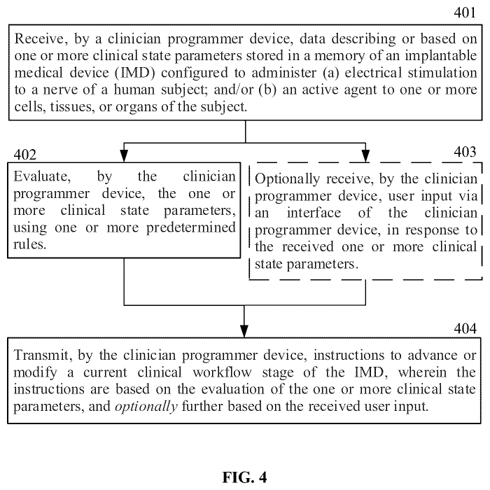

FIG. 5 is a is a block diagram of various example system components, capable of being used along the lines as described in example implementations in accordance with aspects of the present disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary aspects according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

The present disclosure relates to improvements to functional sensing for implantable stimulation systems, such as, e.g., IPGs, and, in some aspects, to methods for energy-efficient cooperation between implanted sensors and external sensing devices.

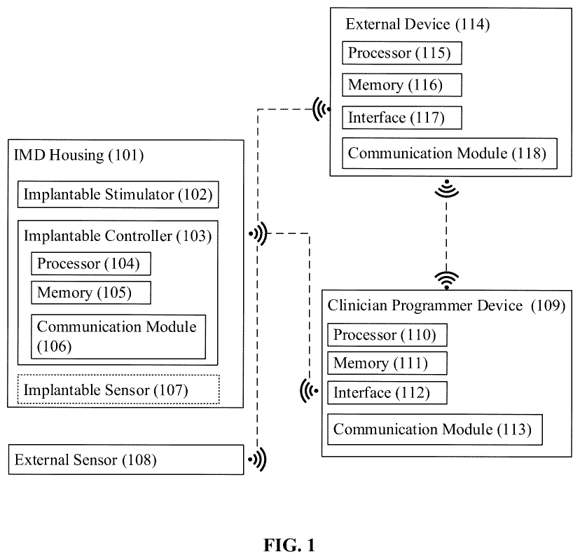

FIG. 1 is a block diagram of an exemplary system according to the present disclosure. This particular example illustrates an IMD comprising a housing 101, containing an implantable stimulator (102) (e.g., an IPG), an implantable controller 103 (e.g., comprising a processor (104), and a memory (105) comprising software code that when executed controls the stimulation system or components thereof). The IMD further includes a communications module (106), e.g., to allow for wireless or wired communication between the IMD and any of the other components of the system shown in this figure or described elsewhere in the present application. In this case, the IMD housing (101) is shown to further include an optional implantable sensor (107) within the housing (101). Implantable sensors (107) may be integrated into an implanted stimulation system, placed on or connected to a housing (101) of an implanted stimulation system, or be implanted separately (e.g., in a separate housing, configured for wireless or wired communication with the controller (103) of the IMD. This block diagram further illustrates an external sensor (108) capable of wirelessly communicating with one or more components of the IMD.

In this example, the IMD is shown to be in wireless communication with one implantable sensor (107) and one external sensor 108. Wireless communication may be enabled by a Bluetooth, near-field communication (“NFC”), or other wireless communication protocol using a wireless modem integrated into the stimulation system. In this example, the wireless modem is envisioned as a component of the implantable controller 103 integrated into the housing 101 of the stimulation system. In other aspects, one or more sensors may be communicatively linked to the implantable controller 103 via a physical connection (e.g., one or more implantable sensors 106 may be connected to the housing 101 and communicatively linked to the implantable controller 103 via electrical leads).

This figure further illustrates the use of an electronic device 110, in this case a wearable device 110a, in a smart watch form factor. The electronic device 110 is shown to be in wireless communication with two implantable sensors 106 and two external sensors 108, as well as with a cloud-based infrastructure 110 that provides a communication link to a remote server 109. The electronic device 110 may be configured to connect to a remote server 109 in order to obtain or transmit information (e.g., biometric parameters of the subject, or stimulation parameters to be used by the stimulation system). For example, the server may be operated by a medical professional (e.g., a clinician programmer) responsible for medical treatment of the subject. The medical professional may use the remote server to view and/or adjust one or more stimulation parameters (e.g., they may set or adjust a pulse frequency, width, amplitude, and/or duty cycle, of the electrical stimulation) or set conditions for when stimulation should be applied (e.g., one or more biometric parameter thresholds that trigger a therapeutic intervention, such as the activation of stimulation or an increase/decrease in stimulation).

In some aspects, the electronic device 110 may be configured to upload historical data to the remote server 109, such as historical biometric parameter levels of the subject, recorded medical events (e.g., onset or duration and/or timing of tachycardia, a seizure, or an apnea/hypopnea event). Such data may be used by a medical professional to monitor and/or evaluate the effect of stimulation parameters, so that therapy may be personalized for the subject.

In some aspects, the electronic device will comprise a controller capable of setting or modifying one or more parameters of the implanted stimulation system. It is envisioned that this functionality may allow the electronic device 110 to act as a master or primary controller of the implanted stimulation system (when available), with the implantable controller 103 serving as a secondary or backup controller to maintain operation (e.g., when the electronic device 110 is not in proximity or otherwise unavailable to the subject).

As illustrated by this figure, the electronic device 110 and the implantable controller 103 may each be in communication with one or more implantable sensors 106 and/or external sensors 108. In some aspects, both devices may maintain separate connections to one or more of the same sensors. However, it is envisioned that energy may be conserved by reducing the number of connections. For example, when the electronic device 110 is available (e.g., detected based on proximity or by the establishment of a communications link with the implantable controller 103), the implantable controller 103 may disable communication with one or more implantable sensors 106 or external sensors 108 in order to conserve the battery of the implanted stimulation system, with the electronic device 110 then taking over collection of sensor data. The electronic device 110 may establish a connection (or maintain a connection) with one or more of the implantable sensors 106 or external sensors 108 that were previously linked with the implantable controller 103. In other aspects, the electronic device 110 may use additional or alternative sensors as a replacement for one or more sensors disabled by the implantable controller 103. For example, the implantable controller 103 may disable an internal IMU used to detect the subject's respiration rate, and the electronic device 110 may obtain functionally equivalent data from an alternative external sensor 108. The electronic device 110 may use the sensor data collected from one or more of the implantable sensors 106 or external sensors 108 to determine one or more biometric parameters of the subject, and to determine whether stimulation is needed or if any stimulation parameters should be adjusted. When it is determined that stimulation is needed (of if stimulation parameters should be adjusted), the electronic device 110 may communicate any such instructions or commands to the implantable controller 103 in order to trigger stimulation according to the selected parameters.

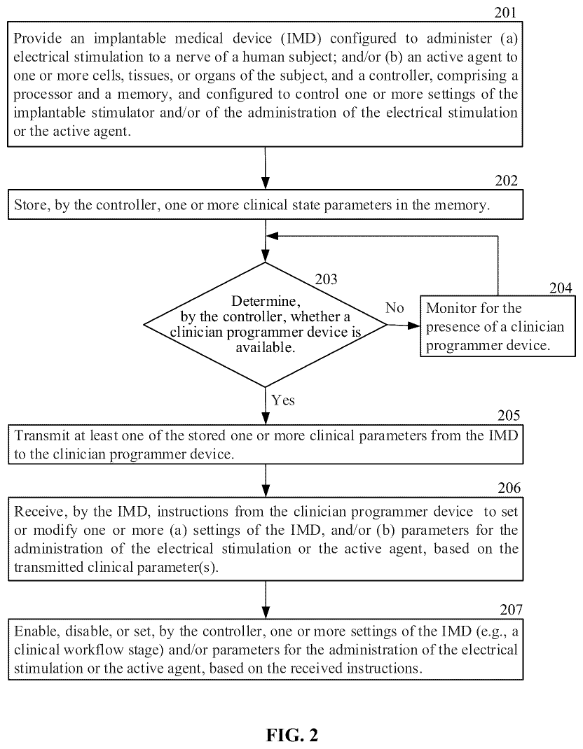

FIG. 2 is a flowchart illustrating an exemplary method according to the disclosure. In this case, the method relates to a processing workflow for controlling an IMD (e.g., setting or enabling/disabling parameters for stimulation or other IMD settings, or advancing or modifying a clinical workflow stage) based on a stored clinical state parameters. In this case, the method begins with a subject being provided with an IMD configured to administer (a) electrical stimulation to a nerve of a human subject; and/or (b) an active agent to one or more cells, tissues, or organs of the subject, and a controller, comprising a processor and a memory, and configured to control one or more settings of the implantable stimulator and/or of the administration of the electrical stimulation or the active agent (201) Next, one or more clinical state parameters may be stored in the memory (202). Clinical state parameters include, without limitation, parameters indicative of or based upon a status, event, or activity performed by the IMD or a subject in which the IMD is or was implanted. Clinical state parameters may be stored in memory, e.g., as a numerical, Boolean, string or other value. In some aspects, the clinical state parameter may comprise a date of an event (a date of a clinical visit by the subject, a date of fitting with the IMD, etc.). The IMD may be configured to determine whether a clinician programmer device is available (203) and/or to monitor for the presence of a clinician programmer device (204). These functions may be performed using any of the steps of parameters described herein (e.g., an IMD may monitor for the presence of a wireless connection advertised by the clinician programmer device). Alternatively, an IMD may be manually connected to a clinician programmer device (e.g., by a user using an interface of the IMD or on the clinician programmer device). After a clinician programmer device is detected or otherwise made available, one or more of the stored clinical state parameters may be transmitted from the IMD to the clinician programmer device (205). The clinician programmer device may use the received parameters to generate instructions to set or modify one or more (a) settings of the IMD, and/or (b) parameters for the administration of the electrical stimulation or the active agent, based on the transmitted clinical parameters. Instructions may be generated automatically (e.g., based on predetermined rules), and/or based on manual entry of instructions by a clinician programmer (e.g., via a user interface of the clinician programmer device). These instructions may be transmitted to the IMD, directly or indirectly. Once received (206), the IMD may in turn enable, disable, or set, by the controller, one or more settings of the IMD (e.g., a clinical workflow stage) and/or parameters for the administration of the electrical stimulation or the active agent, based on the received instructions.

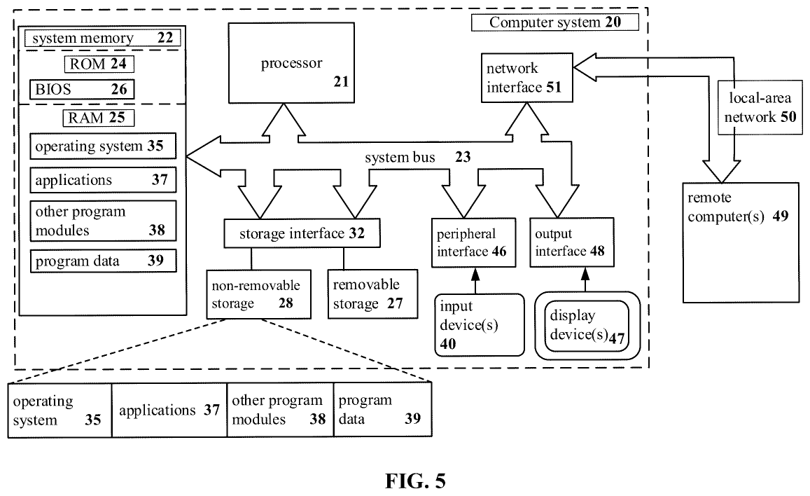

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. FIG. 5 is a block diagram illustrating an example of a computer system 20 which may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain aspects of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or “exemplary” language (e.g., “such as”) provided herein is intended merely to better illuminate the present disclosure and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A system for electronic patient care, comprising:
an implantable medical device (“IMD”) configured to administer (a) electrical stimulation to a nerve of a human subject; and/or (b) an active agent to one or more cells, tissues, or organs of the subject; and
a controller, comprising a processor and a memory, and configured to control one or more settings of the IMD;
wherein the controller is configured to
store one or more clinical state parameters in the memory, and to allow the one or more clinical state parameters to be read, set, or modified by a clinician programmer device, and to
enable, disable, or set the one or more settings of the IMD, and/or the one of more parameters of the administration of the electrical stimulation or the active agent, based on the stored one or more clinical state parameters.

2. The system of claim 1, wherein the one or more clinical state parameters comprise:
a) a parameter indicating or based on an IMD battery level or percentage;
b) a parameter indicating whether the IMD is currently administering the electrical stimulation to a nerve of a subject;
c) a parameter indicating whether the IMD is currently administering the active agent to one or more cells, tissues, or organs of the subject;
d) a parameter indicating whether the IMD is implanted in the human subject;
e) a parameter indicating a date and/or time of an initial or a previous clinical visit by the subject:
f) a parameter indicating a date and/or time when the IMD was implanted in the subject, or a length of time since the IMD was implanted in the subject;
g) a parameter indicating a date and/or time when the subject is due or scheduled for a subsequent clinical visit;
h) a parameter indicating a date and/or time when the subject engaged in a fitting session for the IMD;
i) a parameter indicating a date and/or time when the IMD is due or scheduled for maintenance or replacement;
j) one or more parameters of the electrical stimulation currently or previously administered to the subject, by the IMD, wherein the parameters comprise a pulse frequency, width, amplitude, and/or duty cycle, of the electrical stimulation, and optionally timing or duration information for the electrical stimulation currently or previously administered to the subject;
k) one or more parameters of the active agent currently or previously administered to the subject, by the IMD, wherein the parameters comprise an amount, concentration, dosage schedule or timing, for the currently or previously administered active agent;
l) one or more parameters indicating or based on a location of the IMD;
m) one or more parameters indicating or based on a location, pairing status, availability, or proximity, of a clinician programmer device; and/or
n) one or more parameters of a wired or wireless network connection that is active or available to the IMD.

3. The system of claim 1, wherein the controller is further configured to:
enable, disable, or set the one or more settings of the IMD, and/or the one or more parameters of the administration of the electrical stimulation or the active agent, to the subject, based on the stored one or more clinical state parameters indicating that a wireless network connection to a clinician programmer device is currently active.

4. The system of claim 1, wherein the controller is further configured to:
enable, disable, or set the one or more settings of the IMD, and/or the one or more parameters of the administration of the electrical stimulation or the active agent, to the subject, based on the stored one or more clinical state parameters indicating that a fitting session for the IMD was previously performed.

5. The system of claim 1, wherein the controller is further configured to:
enable, disable, or set the one or more settings of the IMD, and/or the one or more parameters of the administration of the electrical stimulation or the active agent, to the subject, based on the stored one or more clinical state parameters indicating that the IMD is in proximity to or wirelessly connected to, a clinician programmer device.

6. The system of claim 1, wherein the controller is further configured to:
enable, disable, or set the one or more settings of the IMD, and/or the one or more parameters of the administration of the electrical stimulation or the active agent, to the subject, based on the stored one or more clinical state parameters indicating that the battery level and/or percentage of the IMD is above, below, or within a predetermined range or threshold.

7. The system of claim 1, wherein the one or more clinical state parameters stored in the memory of the controller comprise at least one parameter indicating or based on a clinical workflow stage, and the controller is further configured to enable, disable, or set the one or more settings of the IMD, and/or the one or more parameters of the administration of the electrical stimulation or the active agent, based on the clinical workflow stage.

8. The system of claim 7, wherein the IMD is configured to wirelessly advertise or communicate the stored clinical workflow stage, to the clinician programmer device, and to allow the clinician programmer device to modify or advance the stored clinical workflow stage.

9. The system of claim 1, wherein the controller is further configured to:
generate a text, audio, and/or visual notification that the IMD is in need of maintenance or replacement, based on the stored one or more clinical state parameters, optionally wherein the text, audio, and/or visual notification is provided by an interface of the clinician programmer device or an external electronic device.

10. The system of claim 1, wherein the controller is integrated into the IMD, and further configured to:
transmit data based on or comprising the one or more clinical state parameters, to an external electronic device, optionally wherein the electronic device is a clinician programmer device, a smart phone, a smart watch, a computer, or a portable or stationary external controller, or a remote server; and to
allow modification of the one or more clinical state parameters, by the external electronic device.

11. The system of claim 1, wherein the controller is integrated into the IMD, and further configured to:
transmit data based on or comprising the one or more clinical state parameters, to a clinician programmer device configured to display a set of clinical workflow stages via an interface of the clinician programmer device, wherein the set is based on the transmitted data; and
enable, disable, or set the one or more settings of the IMD, and/or the one or more parameters of the administration of the electrical stimulation or the active agent, when a command to execute a selected clinical workflow stage is received from the clinician programmer device.

12. A method for electronic patient care, comprising:
providing an implantable medical device (IMD) configured to administer (a) electrical stimulation to a nerve of a human subject, or (b) an active agent to one or more cells, tissues, or organs of the subject, and a controller, comprising a processor and a memory, and configured to control one or more settings of the IMD;
storing, by the controller, one or more clinical state parameters in the memory, and
enabling, disabling, or setting the one or more settings of the IMD, and/or the one or more parameters of the administration of the electrical stimulation or the active agent, based on the stored one or more clinical state parameters.

13. The method of claim 12, wherein the one or more clinical state parameters comprise:
a) a parameter indicating or based on an IMD battery level or percentage;
b) a parameter indicating whether the IMD is currently administering the electrical stimulation to a nerve of a subject;
c) a parameter indicating whether the IMD is currently administering the active agent to one or more cells, tissues, or organs of the subject;
d) a parameter indicating whether the IMD is implanted in the human subject;
e) a parameter indicating a date and/or time of an initial or previous clinical visit by the subject;
f) a parameter indicating a date and/or time when the IMD was implanted in the subject, or a length of time since the IMD was implanted in the subject;
g) a parameter indicating a date and/or time when the subject is due or scheduled for a subsequent clinical visit;
h) a parameter indicating a date and/or time when the subject engaged in a fitting session for the IMD;
i) a parameter indicating a date and/or time when the IMD is due or scheduled for maintenance or replacement:
j) one or more parameters of the electrical stimulation currently or previously administered to the subject, by the IMD, wherein the parameters comprise a pulse frequency, width, amplitude, and/or duty cycle, of the electrical stimulation, and optionally timing or duration information for the electrical stimulation currently or previously administered to the subject;
k) one or more parameters of the active agent currently or previously administered to the subject, by the IMD, wherein the parameters comprise an amount, concentration, dosage schedule or timing, for the currently or previously administered active agent;
l) one or more parameters indicating or based on a location of the IMD;
m) one or more parameters indicating or based on a location, pairing status, availability, or proximity, of a clinician programmer device; and/or
n) one or more parameters of a wired or wireless network connection that is active or available to the IMD.

14. The method of claim 12, further comprising:
reading, setting, or modifying the one or more stored clinical state parameters, by a clinician programmer device.

15. The method of claim 12, further comprising:
enabling, disabling, or setting, by the controller, the one or more settings of the IMD, and/or the one or more parameters of the administration of the electrical stimulation or the active agent, to the subject, based on the stored one or more clinical state parameters indicating that a wireless network connection to a clinician programmer device is currently active.

16. The method of claim 12, further comprising:
enabling, disabling, or setting, by the controller, the one or more settings of the IMD, and/or the one or more parameters of the administration of the electrical stimulation or the active agent, to the subject, based on the stored one or more clinical state parameters indicating that a fitting session for the IMD was previously performed.

17. The method of claim 12, further comprising:
enabling, disabling, or setting, by the controller, the one or more settings of the IMD, and/or the one or more parameters of the administration of the electrical stimulation or the active agent, to the subject, based on the stored one or more clinical state parameters indicating that the IMD is in proximity to or wirelessly connected to, a clinician programmer device.

18. The method of claim 12, further comprising:
enabling, disabling, or setting, by the controller, the one or more settings of the IMD, and/or the one or more parameters of the administration of the electrical stimulation or the active agent, to the subject, based on the stored one or more clinical state parameters indicating that the battery level and/or percentage of the IMD is above, below, or within a predetermined range or threshold.

19. The method of claim 12, wherein the one or more clinical state parameters stored in the memory of the controller comprise at least one parameter indicating or based on a clinical workflow stage, and the method further comprises
enabling, disabling, or setting, by the controller, the one or more settings of the IMD, and/or the one or more parameters of the administration of the electrical stimulation or the active agent, based on the clinical workflow stage.

20. The method of claim 19, wherein the IMD is configured to wirelessly advertise or communicate the stored clinical workflow stage, to the clinician programmer device, and the method further comprises
receiving, by the controller, an instruction to modify or advance the stored clinical workflow stage, from the clinician programmer device.

21. The method of claim 12, further comprising:
generating, by the controller, a text, audio, and/or visual notification that the IMD is in need of maintenance or replacement, based on the stored one or more clinical state parameters. optionally wherein the text, audio, and/or visual notification is provided by an interface of a clinician programmer device or an external electronic device.

22. The method of claim 12, wherein the controller is integrated into the IMD, and the method further comprises:
transmitting, by the controller. data based on or comprising the one or more clinical state parameters, to an external electronic device, and to
receiving, by the controller, instructions to modify the one or more clinical state parameters, from an external electronic device.
